(19) United States
(12) Plant Patent Application Publication
(10) Pub. No.: US 2004/0098776 P1
(43) Pub. Date: May 20, 2004

(54) Ligularia plant named 'Britt-Marie Crawford'

(76) Inventor: James Crawford, Fife (GB)

Correspondence Address: MILLER JOHNSON SNELL CUMMISKEY, PLC, 800 CALDER PLAZA BUILDING, 250 MONROE AVE N W, GRAND RAPIDS, MI, 49503-2250, US

(21) Appl. No.: 10/294,295

(22) Filed: Nov. 14, 2002

(51) Int. Cl. A01H 5/00

(52) U.S. Cl. PLT/263

(57) ABSTRACT

Ligularia dentata ‘Britt-Marie Crawford’ is a new and distinct cultivar of Ragwort with very dark purple brown foliage that remains effective throughout the growing season, and effective as a potted plant or landscape specimen or mass for the bold foliage effect and colorful yellow-orange flower display.

### BACKGROUND OF THE PLANT

The present invention relates to the new and distinct Ragwort plant, botanically known as Ligularia dentata (A. Gray) hybrid, and hereinafter referred to by the cultivar name ‘Britt-Marie Crawford’. The stated cultivar is valued for its unique, deep, dark mahogany-colored, glossy foliage and late summer flower display.

Ligularia dentata ‘Britt-Marie Crawford’ is a seedling selection of Ligularia dentata ‘Othello’ (not patented) discovered by the inventor, Captain James Crawford, in his garden in Fife, Scotland, United Kingdom. The stated plant is named for the late wife of the inventor. The plant has been asexually propagated by tissue culture and division, and produces true to type plants in successive generations in both of the above-mentioned propagation methods, maintaining its distinct and unique characteristics. Application for Breeder's Right has also been made for Ligularia dentata ‘Britt-Marie Crawford’ in the European Union through the Community Plant Variety Office.

### SUMMARY OF THE INVENTION

Ligularia dentata (A. Gray) is native to eastern China and Japan. There are only a few cultivars, and Ligularia stenocephala ‘Little Rocket’ CPVO application number 2002/1174 has patent protection sought by the Community Plant Variety Office. ‘Britt-Marie Crawford’ is different from all other Ligularia in that it has very dark purple-brown foliage, which is persistent through the season. Other cultivars, such as Ligularia dentata ‘Desdemona’ (not patented) have some purple foliage for a short time in early spring.

### BRIEF DESCRIPTION OF DRAWINGS

The photographs of the new invention demonstrate the overall appearance of the plant including the unique traits. Light source and direction may cause the appearance of variation in color. The colors are as accurate as reasonably possible with color reproductions.

FIG. 1 shows the foliage of the plant.

FIG. 2 shows the flowers of the plant in a more mature state of flowering.

FIG. 3 shows a close-up view of some younger flowers to demonstrate flower color and form.

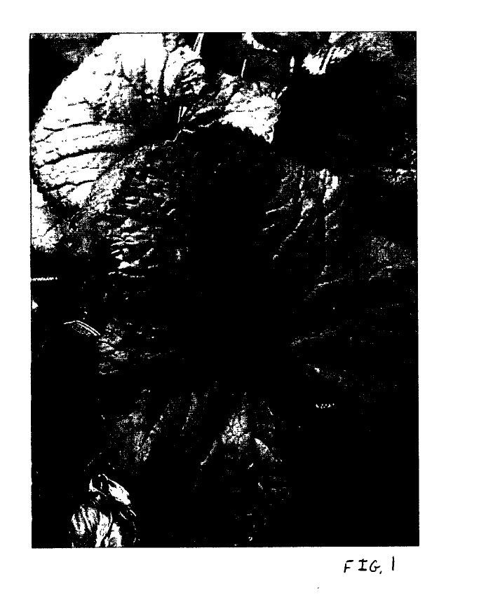

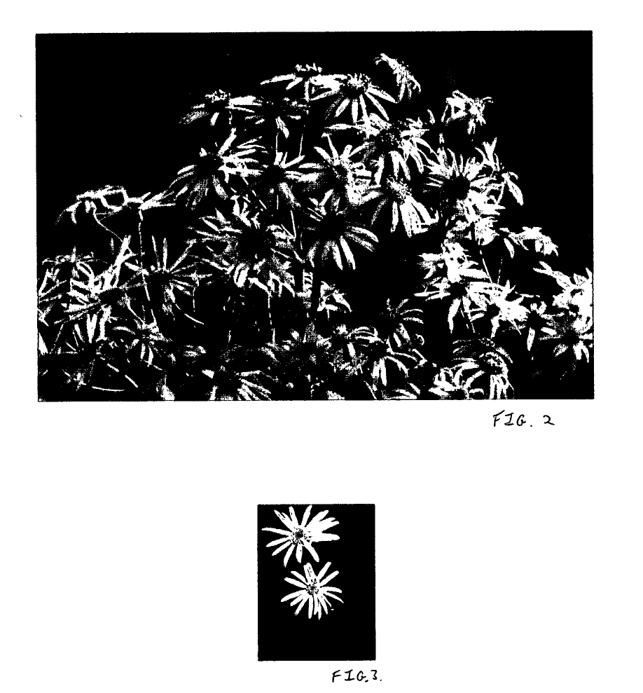

### DETAILED DESCRIPTION OF PLANT VARIETY

The following color description is based on color terminology in accordance with 2001 version of “The Royal Horticultural Society Colour Chart” and is labeled with RHS and the appropriate corresponding reference number and letter, except where common dictionary terms are used. The plant described is a two-year old plant grown in full sun in a garden in Fife, Scotland, United Kingdom.

Botanical classification: Ligularia dentata (A. Gray) hybrid.

Parentage: Ligularia dentata ‘Othello’ seedling.

Propagation: asexual by division or by tissue culture.

Rooting time: approximately three weeks.

Root description: thick fibrous.

Plant habit and form: clump-forming, herbaceous, perennial, with basal, (alternate on stem) long-petioled leaves.

Growth rate: Medium, finishing in a 3-inch pot in approximately 3 months.

Foliage height: 40 to 50 cm.

Foliage spread: 50 to 65 cm.

Leaves: orbicular-reniform, coarsely dentate margined, 20 to 25 per plant, 16 to 18 cm long, 18 to 22 cm wide, apex rounded, base broadly rounded to cordate, abaxial color RHS 183 C, adaxial color between RHS N187 A and RHS N187 B.

Inflorescence: corymbose heads, beginning August and remaining effective for six weeks.

Scape: 80 to 100 cm tall, 70 to 85 cm wide, with approximately 70 to 90 flowers.

Ray petals: approximately 12 to 16 per flower; 22 to 27 mm long, 5 to 7 mm wide; yellow-orange RHS 17 C.

Flower buds at tight petal stage: 1 cm diameter, petals about RHS 17 B.

Disc flowers: approximately 80 per flower, 4 to 5 mm long, 2 mm wide, RHS 13 B when young, darker than RHS 152 A when old.

Anthers: 4 to 6 mm long, less than 1 mm wide, darker than RHS 17 A.

Pollen: RHS 17 A.

Seeds: Achenes, approximately 60 to 100 per flower, glabrous, 6 to 8 mm long, 1 to 1.5 mm wide, less than 1 mm thick, dark brown, nearest RHS 200 A.

The stated plant has not been grown in all possible environments. Some variation in color and size may appear due to variations in temperature, light, and fertilizer, etc. that may affect the phenotype of the plant without any genotypic change.

The stated plant prefers cool moist soils and protection from hot afternoon sun, but will recover from wilt if stressed. The plant is not susceptible to any major pests or diseases, is cold tolerant to at least USDA Zone 4.

1. I claim the new and distinct plant variety, Ligularia dentata ‘Britt-Marie Crawford’, essentially as herein described and illustrated, with yellow-orange flowers and deep dark purple-brown foliage that remains effective throughout the growing season.
